(19) United States
(12) Patent Application Publication
(10) Pub. No.: US 2003/0086923 A1
(43) Pub. Date: May 8, 2003

(54) Method for the prevention and/or treatment of atherosclerosis

(76) Inventors: Carl P. Sparrow, Westfield, NJ (US); John G. Ondeyka, Fanwood, NJ (US); Bruno Tse, Jersey City, NJ (US); Sheo Bux Singh, Edison, NJ (US)

Correspondence Address: MERCK AND CO INC, P O BOX 2000, RAHWAY, NJ, 070650907

(21) Appl. No.: 10/261,710

(22) Filed: Oct. 1, 2002

Related U.S. Application Data
(63) Continuation of application No. 09/716,554, filed on Nov. 20, 2000.
(60) Provisional application No. 60/170,403, filed on Dec. 13, 1999.
(60) Provisional application No. 60/223,049, filed on Aug. 4, 2000.

(51) Int. Cl. A61K 39/395; A61K 38/17

(52) U.S. Cl. 424/143.1; 514/12

(57) ABSTRACT

The instant invention provides a method for raising serum HDL cholesterol levels comprising administering a therapeutically effective amount of an LXR ligand to a patient in need of such treatment. It further provides a method for using an LXR ligand to stimulate expression of the ABC1 gene. LXR ligands can be used for preventing and treating atherosclerosis and related conditions.

### CROSS-REFERENCE TO RELATED APPLICATIONS

This application claims priority to U.S. provisional application S No. 60/170,403, filed Dec. 13, 1999 and U.S. provisional application S No. 60/223,049, filed Aug. 4, 2000, herein incorporated by reference in its entirety.

### BACKGROUND OF THE INVENTION

Recent publications in Nature Genetics, August, 1999 (Young et al, page 316; Bodzioch et al, page 347; Brooks-Wilson et al, page 335, and Rust et al, page 352) showed that humans with mutations in the gene ABC1 have low levels of high density lipoprotein (HDL). Low HDL levels are a risk factor for atherosclerosis, myocardial infarction and related conditions such as ischemic stroke. Therefore, increasing the expression of the ABC1 gene would be expected to increase HDL levels and decrease the occurrence of atherosclerosis, myocardial infarction and related conditions such as ischemic stroke. It has been reported that expression of the ABC1 gene is increased by cholesterol loading of cells (Langmann et al, Biochem. Biophys. Res. Comm., 257, 29-33 (1999)). LXRα is a nuclear receptor that is required for the induction of cholesterol 7α-hydroxylase in mouse liver following cholesterol feeding (Peet et al, Cell, 93, 693-704 (1998)). LXRα and LXRβ are activated by 22-(R)-hydroxycholesterol and other oxysterols (Janowski et al. Proc. Natl. Acad. Sci USA, 96, 266-271 (1999)). As part of the instant invention it was found that LXRα and/or LXRβ cause the induction or regulation of ABC1 expression. We conclude that small molecule ligands of LXR are useful as drugs to increase the expression of ABC1, increase levels of HDL and thereby decrease the risk of atherosclerosis, myocardial infarction and related conditions such as peripheral vascular disease and ischemic stroke.

### SUMMARY OF THE INVENTION

One object of the instant invention is to provide a method for raising serum HDL cholesterol levels comprising administering a therapeutically effective amount of an LXR ligand to a patient in need of such treatment.

Another object is to provide a method for stimulating the expression of the ABC1 gene which comprises administering an effective amount of an LXR ligand to a patient in need of such treatment whereby the patient's serum HDL level is increased.

As a further object, methods are provided for preventing or reducing the risk of developing atherosclerosis, as well as for halting or slowing the progression of atherosclerotic disease once it has become clinically evident, comprising the administration of a prophylactically or therapeutically effective amount, as appropriate, of an LXR ligand to a patient who is at risk of developing atherosclerosis or who already has atherosclerotic disease. The method of this invention also serves to remove cholesterol from tissue deposits such as xanthomas and atherosclerotic lesions by hastening the efflux of cholesterol from cells in those lesions. Additional objects will be evident from the following detailed description.

### BRIEF DESCRIPTION OF DRAWINGS

FIG. 1 shows displacement of [³H₂]Compound A from GST-LXRα, with percent inhibition by Compound 1 at μM concentrations. Compound 1 IC₅₀ is 80 nM (calculated Kᵢ˜30 nM).

FIG. 2 shows displacement of [³H₂]Compound A from GST-LXRα, with percent inhibition by Compound 1 at μM concentrations. Compound 1 IC₅₀ is 40 nM (calculated Kᵢ˜14 nM).

FIG. 3 shows LXRα-GAL4 fusion protein transactivation in cultured cells by various concentrations of Compound 1.

FIG. 4 shows LXRβ-GAL4 fusion protein transactivation in cultured cells by various concentrations of Compound 1.

FIG. 5 shows LXRα-GAL4 and LXRβ-GAL4 fusion protein transactivation in cultured cells by various concentrations of Compound 2.

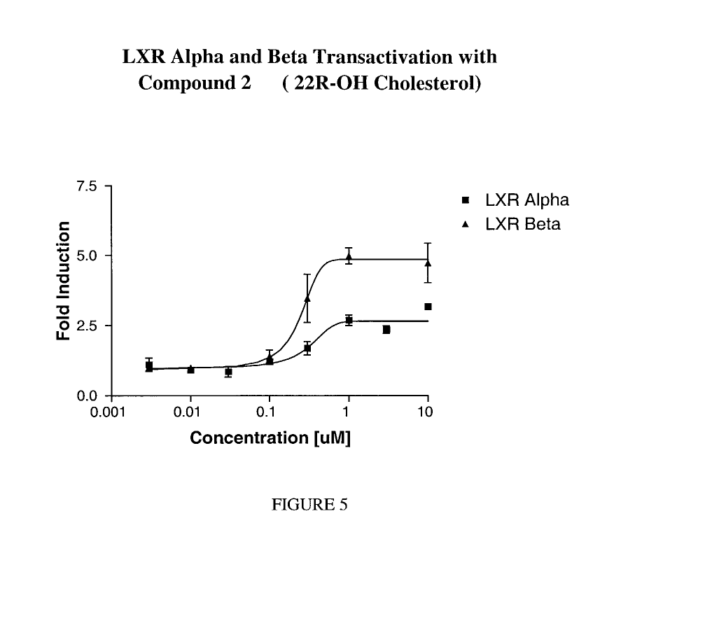

FIG. 6 shows LXRα-GAL4 and LXRβ-GAL4 fusion protein transactivation in cultured cells by various concentrations of Compound 3.

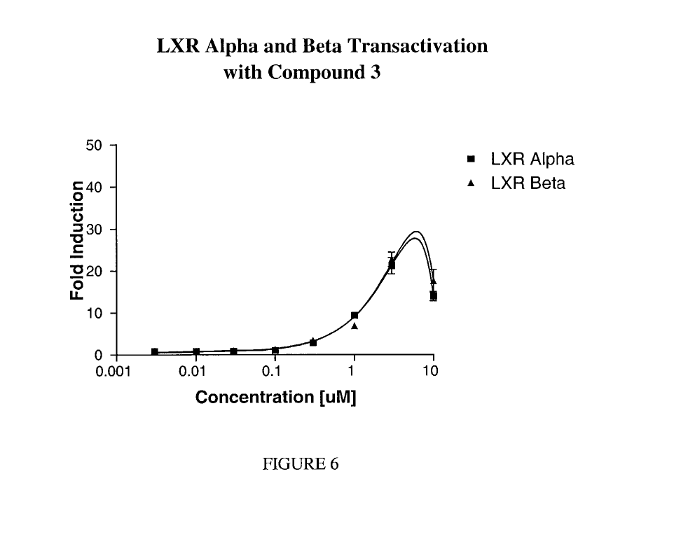

FIG. 7 shows LXRα-GAL4 and LXRβ-GAL4 fusion protein transactivation in cultured cells by various concentrations of Compound 4.

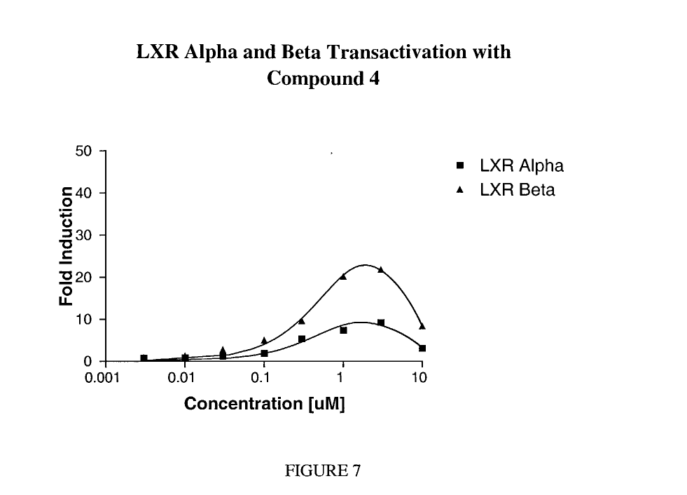

### DETAILED DESCRIPTION OF THE INVENTION

Any patient desiring to increase their HDL cholesterol level may use this treatment. Particularly suitable patients in need of such treatment are those whose HDL level is below the clinically desirable level of HDL cholesterol, i.e, about 40 mg/dl in men and about 50 mg/dl in women.

Atherosclerosis encompasses vascular diseases and conditions that are recognized and understood by physicians practicing in the relevant fields of medicine. Atherosclerotic cardiovascular disease including restenosis following revascularization procedures, coronary heart disease (also known as coronary artery disease or ischemic heart disease), cerebrovascular disease including multi-infarct dementia, and peripheral vessel disease including erectile dysfunction are all clinical manifestations of atherosclerosis and are therefore encompassed by the terms “atherosclerosis” and “atherosclerotic disease.”

An LXR ligand may be administered to prevent or reduce the risk of occurrence, or recurrence where the potential exists, of a coronary heart disease event, a cerebrovascular event, and/or intermittent claudication. Coronary heart disease events are intended to include CHD death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events, and intermittent claudication. It is intended that persons who have previously experienced one or more non-fatal atherosclerotic disease events are those for whom the potential for recurrence of such an event exists.

Accordingly, the instant invention also provides a method for preventing or reducing the risk of a first or subsequent occurrence of an atherosclerotic disease event comprising the administration of a prophylactically effective amount of an LXR ligand to a patient at risk for such an event. The patient may already have atherosclerotic disease at the time of administration, or may be at risk for developing it.

The method of this invention also serves to remove cholesterol from tissue deposits such as atherosclerotic plaques or xanthomas in a patient with atherosclerotic disease manifest by clinical signs such as angina, claudication, bruits, one that has suffered a myocardial infaction or transient ischemic attack, or one diagnosed by angiography, sonography or MRI.

The term LXR includes all subtypes of this receptor and corresponding genes which encode such subtypes. Specifically LXR includes LXRα and LXRβ, and a ligand of LXR should be understood to include a ligand of LXRα or LXRβ. LXRα has been referred to under a variety of names and for purposes of this application LXRα should be understood to mean any gene referred to as LXRα, LXRₐ, LXRalpha, RLD-1, NR1H3 or a gene with homology to accession number U22662 or a protein with homology to a protein encoded by such a polynucleotide. Similarly, LXRβ should be understood to include any gene referred to as LXR_{b}, LXRβ, LXRbeta, NER, NER1, UR, OR-1, R1P15, NR1H2 or a gene with homology to accession number U07132 or a protein with homology to a protein encoded by such a polynucleotide.

The term ligand throughout this application should be understood to include an agonist, partial agonist or antagonist of LXR. The ligand may be selective for LXRα or LXRβ, or it may have mixed binding affinity for both LXRα and LXRβ. Particularly, compounds within the scope of this invention include those which have greater selectivity as determined by binding affinity for LXRα and/or LXRβ receptors than they have for each of the PPARα, γ and δ receptors. More particularly, the compounds included within the scope of this invention have an IC₅₀ less than or equal to 100 nM for at least one of either the LXRα or LXRβ receptors, and have an IC₅₀ equal to or greater than 1 μM for each of the PPARα, PPARγ and PPARδ receptors, and even more particularly they have an IC₅₀ equal to or greater than 10 μM for each of the PPARα, PPARγ and PPARδ receptors. For example, the selectivity of suitable LXR receptor ligands can be determined from IC₅₀ results obtained employing the LXR radioligand competition scintillation proximity assays described below in the Example section, and from PPAR competition binding assays described in Berger J, et al., Novel peroxisome proliferator-activated receptory (PPARγ) and PPARδ ligands produce distinct biological effects, J Biol Chem 274: 6718-6725 (1999), herein incorporated by reference in its entirety.

The term “patient” includes mammals, especially humans, who use the instant active agents for the prevention or treatment of a medical condition. Administering of the drug to the patient includes both self-administration and administration to the patient by another person. The patient may be in need of treatment for an existing disease or medical condition, or may desire prophylactic treatment to prevent or reduce the risk for diseases and medical conditions affected by HDL cholesterol.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. Particularly, the dosage amount of an LXR ligand that a patient receives can be selected so as to achieve the amount of HDL cholesterol raising desired; the dosage a patient receives may also be titrated over time in order to reach a target HDL level.

An effective amount of an LXR ligand in the method of this invention is about 0.01 mg/kg to about 140 mg/kg of body weight per day, or about 0.5 mg to about 7 g per patient per day. For example, adequate elevation of HDL can be accomplished by the administration of about 0.5 mg to about 3.5 mg per patient per day.

It will be understood, however, that the specific dose level for any particular patient will depend upon a variety of factors including the age, body weight, general health, sex, diet, time of administration, route of administration, rate of excretion, drug combination and the severity of the particular HDL deficiency. A consideration of these factors is well within the purview of the ordinarily skilled clinician for the purpose of determining the therapeutically effective or prophylactically effective dosage amount needed to prevent, counter, or arrest the progress of the condition.

An example of LXR ligands suitable for use in the method of this invention is represented by Compounds 1-4 having the following structural formulas:

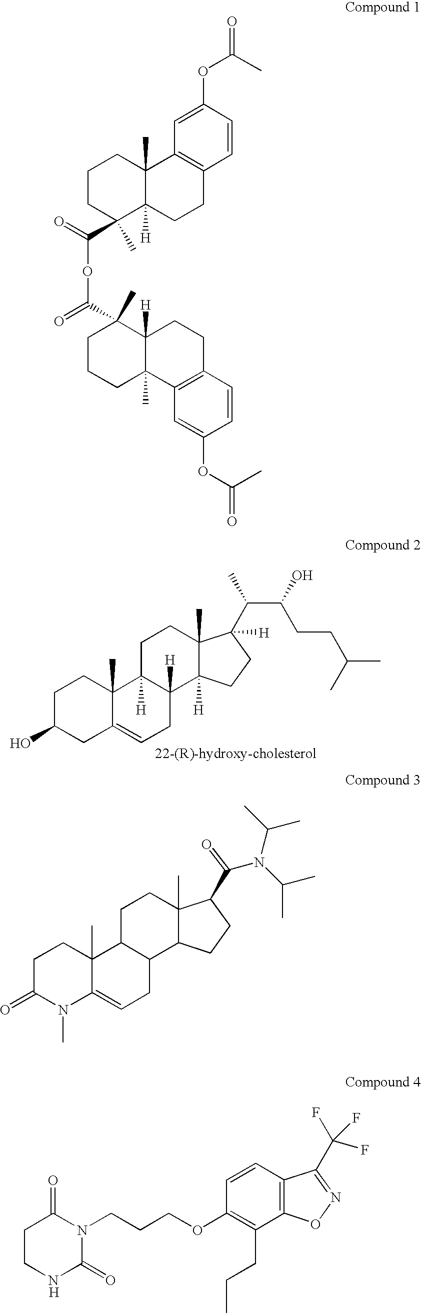

Compound 2 (22(R)-hydroxycholesterol) is available from Sigma-Aldrich. Compound 3 can be prepared as described in Rasmusson et al., J. Med. Chem., v29, p2298 (1986), herein incorporated by reference. Preparation of compounds 1 and 4 (4,5-Dihydro-1-(3-(3-trifluoromethyl-7-propyl-benzisoxazol-6-yloxy)propyl)-2,6-pyrimidinedione) are given in the Examples, below.

In the method of treatment of this invention, the LXR receptor ligands described above may be administered orally, topically, parenterally, by inhalation spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques.

The pharmaceutical compositions of this invention containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. Oral immediate-release and time-controlled release dosage forms may be employed. Tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. One example of a time-controlled release device is described in U.S. Pat. No. 5,366,738. They may also be coated by the technique described in U.S. Pat. Nos. 4,256,108; 4,166,452; and 4,265,874 to form osmotic therapeutic tablets for controlled release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water or miscible solvents such as propylene glycol, PEGs and ethanol, or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more colouring agents, one or more flavouring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. Cosolvents such as ethanol, propylene glycol or polyethylene glycols may also be used. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds useful in the method of treatment of the invention may also be administered in the form of a suppository for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, gels, solutions or suspensions, etc., containing the compound of are employed. For purposes of this application, topical application shall include mouth washes and gargles. Topical formulations may generally be comprised of a pharmaceutical carrier, cosolvent, emulsifier, penetration enhancer, preservative system, and emollient.

The amount of active ingredient that may be combined with the carrier materials to produce a single dosage form will vary depending upon the host treated and the particular mode of administration. For example, a formulation intended for the oral administration of humans may contain from 0.5 mg to 5 g of active agent compounded with an appropriate and convenient amount of carrier material which may vary from about 5 to about 95 percent of the total composition. Dosage unit forms will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg.

One or more additional active agents may be used in combination with the LXR ligands of this invention in a single dosage formulation, or may be administered to the patient in a separate dosage formulation, which allows for concurrent or sequential administration of the active agents. The additional active agent or agents can be lipid altering compounds such as HMG-CoA reductase inhibitors, or agents having other pharmaceutical activities, or agents that have both lipid-altering effects and other pharmaceutical activities. Examples of HMG-CoA reductase inhibitors include statins in their lactonized or dihydroxy open acid forms and pharmaceutically acceptable salts and esters thereof, including but not limited to lovastatin (see U.S. Pat. No. 4,342,767); simvastatin (see U.S. Pat. No. 4,444,784); dihydroxy open-acid simvastatin, particularly the ammonium or calcium salts thereof; pravastatin, particularly the sodium salt thereof (see U.S. Pat. No. 4,346,227); fluvastatin particularly the sodium salt thereof (see U.S. Pat. No. 5,354,772); atorvastatin, particularly the calcium salt thereof (see U.S. Pat. No. 5,273,995); cerivastatin, particularly the sodium salt thereof (see U.S. Pat. No. 5,177,080), and nisvastatin also referred to as NK-104 (see PCT international publication number WO 97/23200). Additional active agents which may be employed in combination with an LXR ligand include but are not limited to HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT1 and -2; microsomal triglyceride transfer protein (MTP) inhibitors; probucol; niacin; cholesterol absorption inhibitors such as SCH-58235 also known as ezetimibe and 1-(4-fluorophenyl)-3(R)-[3(S)-(4-fluorophenyl)-3-hydroxypropyl)]-4(S)-(4-hydroxyphenyl)-2-azetidinone, which is described in U.S. Pat. Nos. 5,767,115 and 5,846,966; bile acid sequestrants; LDL (low density lipoprotein) receptor inducers; platelet aggregation inhibitors, for example glycoprotein IIb/IIIa fibrinogen receptor antagonists and aspirin; human peroxisome proliferator activated receptor gamma (PPARγ) agonists including the compounds commonly referred to as glitazones for example troglitazone, pioglitazone and rosiglitazone and, including those compounds included within the structural class known as thiazolidinediones as well as those PPARγ agonists outside the thiazolidinedione structural class; PPARα agonists such as clofibrate, fenofibrate including micronized fenofibrate, and gemfibrozil; PPAR dual α/γ agonists; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; calcium channel blockers such as nifedipine and diltiazam; endothelian antagonists; agents other than LXR ligands that enhance ABC1 gene expression; bisphosphonate compounds such as alendronate sodium; and cyclooxygenase-2 inhibitors such as rofecoxib and celecoxib.

Compound A is used in the following assays and has the following structural formula:

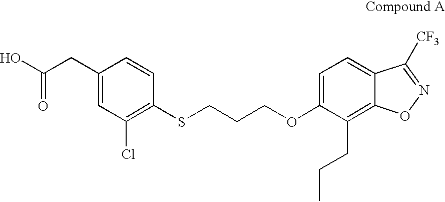

Compound A and related compounds are disclosed along with methods for making them in WO97/28137 (U.S. Ser. No. 08/791,211, filed Jan. 31, 1997) herein incorporated by reference in its entirety.

### EXAMPLE 1

Radioligand Competition Binding Scintillation Proximity Assays:

Preparation of Recombinant Human LXRα and LXRβ:

Human LXRα and LXRβ were expressed as GST-fusion proteins in E. coli. The ligand binding domain cDNAs for human LXRα (amino acids 164-447) and human LXRβ (amino acids 149-455) were subcloned into the pGEX-KT expression vector (Pharmacia). E. coli containing the respective plasmids were propagated, induced, and harvested by centrifugation. The resuspended pellet was broken in a French press and debris was removed by centrifugation. Recombinant human LXR receptors were purified by affinity chromatography on glutathione sepharose and receptor was eluted with glutathione. Glycerol was added to a final concentration of 50% to stabilize the receptor and aliquots were stored at −80° C.

Binding to LXRα:

For each assay, an aliquot of human GST-LXRα receptor was incubated in a final volume of 100 μl SPA buffer (10 mM Tris, pH 7.2, 1 mM EDTA, 10% glycerol, 10 mM Na molybdate, 1 mM dithiothreitol, and 2 μg/ml benzamidine) containing 1.25 mg/ml yttrium silicate protein A coated SPA beads (Amersham Pharmacia Biotech, Inc.), 8.3 μg/ml anti-GST antibody (Amersham Pharmacia Biotech, Inc.) 0.1% non-fat dry milk and 25 nM [³H₂]Compound A (13.4 Ci/mmole), ±test compound. After incubation for ˜16 h at 15° C. with shaking, the assay plates were counted in a Packard Topcount. In this assay the K_{d} for Compound A for LXRα is ≈15 nM.

Binding to LXRβ:

For each assay, an aliquot of human GST-LXRβ ligand binding domain receptor was incubated in a final volume of 100 μl SPA buffer (10 mM Tris, pH 7.2, 1 mM EDTA, 10% glycerol, 10 mM Na molybdate, 1 mM dithiothreitol, and 2 μg/ml benzamidine) containing 1.25 mg/ml yttrium silicate protein A coated SPA beads (Amersham Pharmacia Biotech, Inc.), 8.3 μg/ml anti-GST antibody (Amersham Pharmacia Biotech, Inc.) 0.1% non-fat dry milk and 25 nM [³H2]Compound A (13.4 Ci/mmole), ±test compound. After incubation for ˜16 h at 15° C. with shaking, the assay plates were counted in a Packard Topcount. In this assay the K_{d} for Compound A for LXRβ is ≈10 nM.

Results

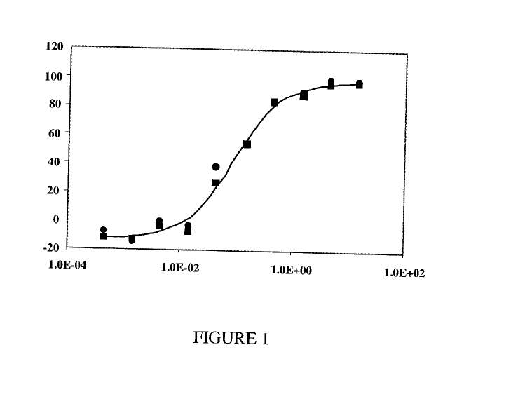

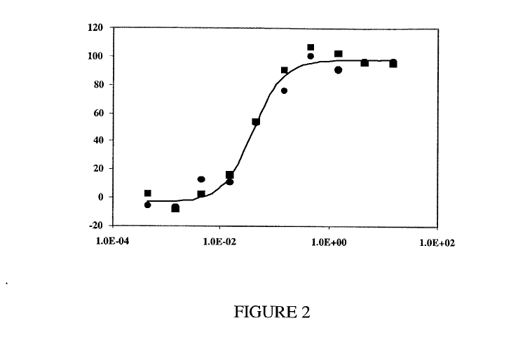

Compound 1 is a ligand for human LXRα and human LXRβ having an IC₅₀=80 nM for the LXRα receptor, and an IC₅₀=40 nM for the LXRβ receptor, as shown in FIGS. 1 and 2. Compound 1 has an IC₅₀ greater than 10 μM in binding assays for human PPARγ, PPARδ and PPARα.

### EXAMPLE 2

Transactivation Assay

Plasmids

Expression constructs were prepared by inserting the ligand binding domain (LBD) of human LXRα and LXRβ cDNAs adjacent to the yeast GAL4 transcription factor DNA binding domain (DBD) in the mammalian expression vector pcDNA3 to create pcDNA3-LXRα/GAL4 and pcDNA3-LXRβ/GAL4, respectively. The GAL4-responsive reporter construct, pUAS(5×)-tk-luc, contained 5 copies of the GAL4 response element placed adjacent to the thymidine kinase minimal promoter and the luciferase reporter gene. The transfection control vector, pEGFP-N1, contained the Green Fluorescence Protein (GFP) gene under the regulation of the cytomegalovirus promoter.

Assay

HEK-293 cells were seeded at 40,000 cells/well in 96 well plates in Dulbecco's modified Eagle medium (high glucose) containing 10% charcoal stripped fetal calf serum (FCS), 100 units/ml Penicillin G and 100 μg/ml Streptomycin sulfate at 37° C. in a humidified atmosphere of 5% CO₂. After 24 h, transfections were performed with Lipofectamine (Gibco-BRL, Gaithersburg, Md.) according to the instructions of the manufacturer. In general, transfection mixes contained 0.002 μg of LXRα/GAL4 or LXRβ/GAL4 chimeric expression vectors, 0.02 μg of reporter vector pUAS(5×)-tk-luc and 0.034 μg of pEGFP-N1 vector as an internal control of transfection efficiency. Compounds were characterized by incubation with transfected cells for 48h across a range of concentrations. Cell lysates were prepared from washed cells using Cell Lysis Buffer (Promega) according to the manufacturer's directions. Luciferase activity in cell extracts was determined using Luciferase Assay Buffer (Promega) in a ML3000 luminometer (Dynatech Laboratories). GFP expression was determined using the Tecan Spectrofluor Plus at excitation wavelength of 485 nm and emission at 535 nm. Luciferase activity was normalized to GFP expression to account for any variation in efficiency of transfection.

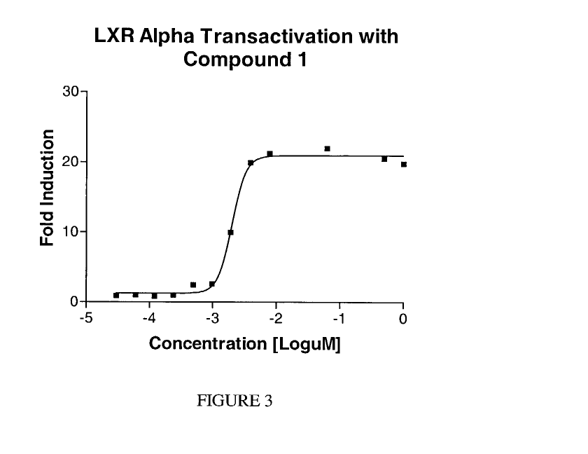

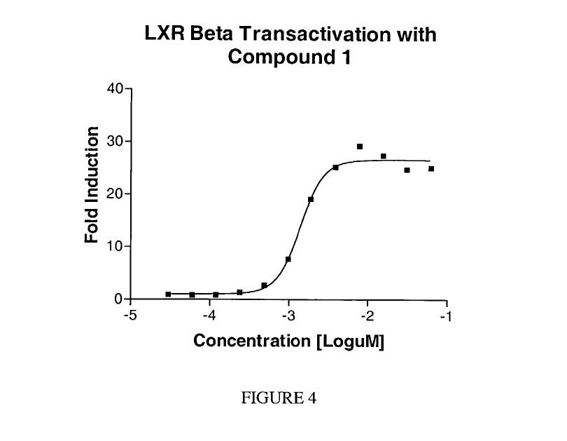

Results with Compound 1 for LXRα transactivation are shown in FIG. 3, and results for LXRβ transactivation are shown in FIG. 4.

### EXAMPLE 3

Induction of ABC1 mRNA levels

Cultured human THP-1 cells were used. All cell culture incubations were performed at 37° C. under 95% air/5% carbon dioxide. Cells were grown in Complete RPMI medium plus 10% FCS (Fetal Calf Serum). Complete RPMI medium is defined as RPMI medium (Sigma Cat.# R8005) containing 0.05 μM β-mercaptoethanol, 1 mM Na Pyruvate, 2 mM L-Glutamine, and Antibiotic-Antimyotic Solution (Sigma Cat.# A9909: 100U/ml Penicillin, 0.1 mg/ml Streptomycin, 0.251l/ml Amphotericin B). Cells were differentiated into macrophages by incubation in Complete RPMI medium plus 10% FCS (Fetal Calf Serum) plus 100 nM tetradecanoyl phorbol acetate for three days. After differentiation, the THP-1 macrophages were incubated in Complete RPMI medium plus 10% FCS (Fetal Calf Serum) with the test LXR agonist. After 6 hours at 37° C., the cells were harvested and total RNA prepared using the phenol/guanidine isothiocyanate method as supplied and described by Molecular Research Center, Inc. (TRI REAGENTφ Cat. No. TR 118). ABC1 mRNA levels in the total RNA were measured using the TaqMan® mRNA quantitation system, following protocols published by the manufacturer (Perkin-Elmer). The oligonucleotide PCR primers used to detect ABC1 were:

GAGGCTCCCGGAGTTGTTG and GTATAAAAGAAGCCTCCGAGCATC

The oligonucleotide probe used was:

6FAM-AAACTTTAACAAATCCATTGTGGCTCGCCTGT-TAMRA

ABC1 mRNA levels in each sample were normalized to the mRNA levels for the 23 kDa highly basic protein. The oligonucleotide PCR primers used to detect the 23 kDa highly basic protein were:

GCTGGAAGTACCAGGCAGTGA and ACCGGTAGTGGATCTTGGCTTT

The oligonucleotide probe used was:

VIC-TCTTTCCTCTTCTCCTCCAGGGTGGCT-TAMRA

The results from this experiment for Compound 1 and Compound 2 (22-(R)-hydroxycholesterol) are as follows, with dimethyl sulfoxide (DMSO) control:

| | Fold Induction of ABC1 | P Value vs |
|---|---|---|
| Compound | mRNA (Mean ± SEM) | DMSO Control |
| 15 μM 22-(R)- | 6.7 ± 1.2 | 0.008 |
| hydroxycholesterol | | |
| (Compound 2) | | |
| 0.10 μM Compound 1 | 6.9 ± 0.6 | 0.0009 |

### EXAMPLE 4

Stimulation of Cholesterol Efflux from Cultured Cells

Patients with Tangier disease have mutations in the ABC1 gene and have very low HDL levels. Cultured cells from these patients are defective in cholesterol efflux (Francis et al. (1995) J. Clin. Invest. 96:78-87). Therefore, induction of ABC1 mRNA should lead to increased cholesterol efflux and increased HDL levels (Francis et al. (1999) Clinica Chimica Acta 286:219-230). TIIP-1 cells were used to determine whether induction of ABC1 mRNA by Compound 1 would increase cholesterol efflux. Specifically, cultured human THP-1 cells were stimulated to differentiate into macrophages by incubation with 100 nM tetradecanoyl phorbol acetate for three days, as described above. After differentiation, the TUP-1 macrophages were labeled with ³H-cholesterol by incubation for 24 hours with 10 μCi/ml ³H-cholesterol in Complete RPMI media containing 0.1% lipid-free bovine serum albumin (BSA) plus 100 nM tetradecanoyl phorbol acetate. After labeling, the cells were incubated for 24 hours in Complete RPMI media with 0.1% lipid-free bovine serum albumin (BSA) to allow for equilibration of cellular cholesterol pools. Cholesterol efflux was then measured over the following 24 hours by incubating the cells in Complete RPMI media containing 10 μg/ml of apoA-I as cholesterol acceptor, and either Compound 1 or DMSO as control. The results, presented in the table below as mean±SEM of quadruplicates, show that Compound 1 significantly increased cholesterol efflux from cultured cells.

| | | |
|---|---|---|
| | Cholesterol efflux | P value |
| Compound added | (% of cell contents) | vs DMSO |

| | | |
|---|---|---|
| DMSO control | 9.9 ± 0.5 | |
| Compound 1 (0.02 μM) | 14.6 ± 0.5 | 0.001 |
| Compound 1 (2.5 μM) | 17.1 ± 0.6 | 0.0001 |

### EXAMPLE 5

Stimulation of Cholesterol Efflux from Cholesterol-Loaded Cells

To determine if LXR agonists could increase cholesterol efflux in cells that were loaded with excess cholesterol, THP-1 cells were loaded with cholesterol using acetylated LDL prior to the assay of cholesterol efflux Specifically, cultured human THP-1 cells were stimulated to differentiate into macrophages as described above. After differentiation, the THP-1 macrophages were loaded with cholesterol and simultaneously labeled with ³H-cholesterol using acetylated LDL containing ³H-cholesterol essentially as described by Kritharides et al. (1998) Arterioscler Thromb Vasc Biol. 18:1589-1599. After 24 hours of incubation with 100 μg/ml of acetylated LDL labelled with ³H-cholesterol, the cells were incubated in Complete RPMI medium containing 0.1% lipid-free BSA for 24 hours to allow for equilibration of cellular cholesterol pools. Cholesterol efflux was then measured over the following 24 hours by incubating the cells in Complete RPMI medium with either apoA-I or HDL as cholesterol acceptor, and with either 1 μM Compound 1 or DMSO as control. The data in the table below (mean±SEM of quadruplicates) shows that Compound 1 increased cholesterol efflux from cholesterol-loaded cells, whether the acceptor was apoA-I or HDL.

| Acceptor in | Cholesterol efflux (% of cell contents) | | P value |
|---|---|---|---|
| medium | Compound 1 (1 μM) | DMSO control | vs DMSO |
| 10 μg/ml apoA-I | 10.3 ± 0.4 | 6.4 ± 0.3 | 0.0003 |
| 25 μg/ml HDL | 18.6 ± 0.7 | 14.9 ± 0.5 | 0.006 |

### EXAMPLE 6

The following are the indicated assay results for compounds 2, 3 and 4.

Compound 2

Cholesterol Efflux Data: 5 μM of Compound 2 stimulated cholesterol efflux 57% (p<0.001), whereas the isomer 22-(S)-hydroxy-cholesterol was inactive on LXR and did not stimulate cholesterol efflux at all.

| Compound 3 | |
|---|---|
| LXRα Binding IC₅₀ (nM): | 112 |
| LXRβ Binding IC₅₀ (nM): | 442 |
| Cholesterol Efflux EC₅₀ (nM): | 120 |
| Compound 4 | |
| LXRα Binding IC₅₀ (nM): | 1 |
| LXRβ Binding IC₅₀ (nM): | 72 |
| LXRα transactivation EC₅₀ (nM): | 183 |
| LXRβ transactivation EC₅₀ (nM): | 360 |
| Cholesterol Efflux EC₅₀ (nM): | 63 |

HDL-Raising Activity: Hamsters were given Compound 4 by oral gavage at 10 mg per kg body weight, twice per day, for seven days. Blood was then collected and serum HDL cholesterol measured. Compared to a control group given gavage vehicle only, this compound increased HDL cholesterol levels by 21% (p=0.01).

### EXAMPLE 7

Preparation of Compound 1

Podocarpic acid (550 mg) was dissolved in 2 ml of acetic anhydride in a 10 ml flask and heated to reflux (150° C.) for 30 minutes and cooled. The reaction was analyzed by HPLC. The major product was the mixed anhydride and about 1% of the reaction mixture was the acetate dimer Compound 1. The solvent was blown off under nitrogen and the resultant oil was charged to a 200 cc Sephadex LH20 column in MeOH (methanol). Compound 1 eluted in cuts 75-80 (2 ml each, 0.8 cv). The mixed anhydride eluted in cuts 80-100. Cuts 75-80 were dried down and dissolved in 300 ul CH₃CN and loaded on a semi-preparative Zorbax RX-C8 column. The column was eluted with a 40-min gradient of 50 to 90% aqueous CH₃CN at 4 mL per min. One-minute fractions were collected. Compound 1 eluted at 35 min. The pooled fractions gave 0.7 mg of Compound 1. Mass spectral analysis of this compound gave a molecular weight of 614 amu and molecular formula of C₃₈H₄₆O₇.

Mass spectral data: Found: 632.3620; Calculated: 632.3587; Formula: C₃₈H₅₀NO₇; Assignment: [M⁺NH4].

¹H NMR data: (δ, 500 MHz, CDCl₃): δ 1.14(1H, dt, 13.5,4.0 Hz), 1.20(3H,s), 1.39(3H,s), 1.44(1H,dt, 13.5, 4.0 Hz), 1.63(1H, d, 12.5), 1.67(1H, m), 2.0(1H, d, 14.0H ), 2.05(1H,m), 2.2(1H, dd,13.5, 6.0 Hz), 2.25(1H,d, 12 Hz), 2.28(3H,s), 2.30(1H,d, 13.5 Hz), 2.80(1H, ddd, 13.0, 12.5,6.5 Hz), 2.95(1H, dd, 16.5,5.0 Hz), 6.83(1H, dd, 8.0,2.0 Hz), 6.96(1H,d, 2.0 Hz), 7.05(1H,d, 8.0 Hz).

Equipment: Mass spectra were recorder on an LCQ (LC-MS-ESI, Liquid chromatography-Electrospray ionization) and exact mass measurements were recorded on a Finnigan NewStar FTMS mass spectrometer. ¹H spectra were recorded in either CDCl₃ or CD₃OD on a Varian Unity 500 NMR Spectrometer operating at 500 MHz for ¹H. Chemical shifts are given in ppm relative to tetramethylsilane (TMS) at zero ppm using the respective solvent peaks as an internal standard.

### EXAMPLE 8

Preparation of Compound 4 (4,5-Dihydro-1-(3-(3-trifluoromethyl-7-propyl-benzisoxazol-6-yloxy)propyl)-2,6-pyrimidinedione)

Step A: Preparation of 2,4-dihydroxy-3-propyl-trifluoro-acetophenone

A solution of 2-propylresorcinol (5.0 grams) and trifluoroacetic anhydride (9.6 mL) in 1,2-dichloroethane (30.0 mL) was treated with aluminum chloride (4.38 grams). This mixture was stirred overnight. The reaction mixture was partitioned between methylene choride and water. The organic phase was dried over sodium sulfate and filtered. The solvent was evaporated and the resulting solid was recrystallized using methylene chloride and cyclohexane (1:1) to give 2,4-dihydroxy-3-propyl-trifluoro-acetophenone.

1H NMR (CDC13) δ 7.59 (d, 1H), 6.24 (d, 1H), 5.92 (s, 1H), 2.63 (t, 2H), 1.74 (s, 1H), 1.58 (m, 2H), 0.98 (t, 3H) ppm.

STEP B: Preparation of 3-trifluoromethyl-7-propyl-6-hydroxy-benzisoxazole

A mixture of 2,4-dihydroxy-3-propyl-trifluoroacetophenone (2.5 grams), sodium acetate (4.18 grams), hydroxylamine hydrochloride (3.59 grams) and methanol (80 mL) was refluxed overnight. The solvent was then evaporated and the resulting solid was partitioned in ethyl acetate and pH 7 buffer. The organic phase was separated and washed with brine. The organic phase was dried over sodium sulfate and the solvent was evaporated to give an oil. The oil was then dissolved in acetic anhydride. The solution was stirred for two hours, then the acetic anhydride was evaporated in vacuo. The residue was partitioned between ethyl acetate and pH 7 buffer and the organic phase was dried over sodium sulfate. The organic phase was evaporated to give an oil. This was dissolved in pyridine and refluxed overnight. The solvent was evaporated in vacuo to give an oil which was chromatographed on silica gel using ethyl acetate and hexane (1:4) to give the titled compound.

¹H NMR (CDCl₃) δ 7.46 (d, 1H), 6.92 (d, 1H), 5.42 (bs, 1H), 2.89 (t, 2H), 1.74 (m, 2H), 0.98 (t, 3H) ppm.

STEP C: Preparation of 3-trifluoromethyl-7-propyl-6-(3-bromopropyloxy)-benzisoxazole

To a DMF solution (50 mL) of 6-hydroxy-7-propyl-3-trifluoromethylbenzisoxazole (5 g, 20.4 mmol) was added 1,3-dibromopropane (10 mL, 98.5 mmol), followed by Cs₂CO₃ (10 g, 30.7 mmol). The mixture was stirred at room temperature overnight. After aqueous work-up (ether) and chromatography, 7.74 g of crude 3-trifluoromethyl-7-propyl-6-(3-bromopropyloxy)-benzisoxazole was obtained, which was used in the next step without further purification.

STEP D: Preparation of 4,5-Dihydro-1-(3-(3-trifluoromethyl-7-propyl-benzisoxazol-6-yloxy)propyl)-2,6-pyrimidinedione

To a DMF solution (20 mL) of 3-trifluoromethyl-7-propyl-6-(3-bromopropyloxy)-benzisoxazole (600 mg) was added 5,6-dihydrouracil (750 mg, 6.58 mmol), followed by Cs₂CO₃ (2.1 g, 6.4 mmol). The mixture was stirred at room temperature overnight. To the mixture was added water (10 mL), followed by TFA (2 mL) at 0° C. The mixture was then purified by HPLC. After concentration in vacuo, 418 mg of 4,5-dihydro-1-(3-(3-trifluoromethyl-7-propyl-benzisoxazol-6-yloxy)propyl)-2,6-pyrimidinedione was obtained.

¹H NMR (C₆D₆): δ 0.95 ppm (3H, t, J=7.3 Hz), 1.69-1.98 (8H, m), 2.92 (2H, t, J=7.6), 3.54 (2H, t, J=6.3), 3.94 (2H, t, J=7.2), 4.05 (1H, br), 6.42-7.20 (2H, 2d, J=8.8). MS: m/z=400 (M+H).

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed:

1. A method for raising serum HDL cholesterol levels comprising adminstering an effective HDL-raising amount of an LXR receptor ligand to a patient in need of such treatment.

2. The method of claim 1, wherein the LXR receptor is an LXRα receptor.

3. The method of claim 1, wherein the LXR receptor is an LXRβ receptor.

4. The method of claim 1, wherein the ligand is an agonist.

5. The method of claim 1, wherein the ligand is an antagonist.

6. The method of claim 1, wherein the ligand is a partial agonist.

7. The method of claim 2, wherein the ligand is an agonist.

8. The method of claim 2, wherein the ligand is an antagonist.

9. The method of claim 2, wherein the ligand is a partial agonist.

10. The method of claim 3, wherein the ligand is an agonist.

11. The method of claim 3, wherein the ligand is an antagonist.

12. The method of claim 3, wherein the ligand is a partial agonist.

13. The method of claim 1 wherein the LXR ligand binds with greater affinity to an LXR receptor than to a PPAR receptor.

14. The method of claim 13 wherein the LXR ligand has an IC₅₀ less than or equal to 100 nM for at least one of an LXR receptor selected from LXRα and LXRβ, and an IC₅₀ equal to or greater than 1 μM for each of the PPARα, PPARγ and PPARδ receptors.

15. The method of claim 14 wherein the LXR ligand has an IC₅₀ equal to or greater than 10 μM for each of the PPARα, PPARγ and PPAR6 receptors.

16. A method for preventing or reducing the risk of developing atherosclerotic disease comprising administering an HDL-raising amount of an LXR receptor ligand to a patient in need of such treatment.

17. A method for treating atherosclerotic disease comprising administering an HDL-raising amount of an LXR receptor ligand to a patient in need of such treatment.

18. A method for preventing or reducing the risk of occurrence or recurrence of an atherosclerotic disease event comprising administering an HDL-raising amount of an LXR receptor ligand to a patient in need of such treatment.

19. A method for stimulating the expression of the ABC1 gene and thereby raising serum HDL cholesterol levels comprising administering an LXR ligand in an amount capable of stimulating expression of the ABC 1 gene to a patient in need of such treatment.

20. A method for stimulating cholesterol efflux comprising administering a therapeutically effective amount of an LXR receptor ligand to a patient in need of such treatment.

21. The compound 4,5-dihydro-1-(3-(3-trifluoromethyl-7-propyl-benzisoxazol-6-yloxy)propyl)-2,6-pyrimidinedione.

22. A compound having the following structural formula [chemical structure]
